(19) United States
(12) Patent Application Publication
(10) Pub. No.: US 2005/0003393 A1
(43) Pub. Date: Jan. 6, 2005

(54) Psychoactive compound associated markers and method of use thereof

(76) Inventors: Erik Gunther, Branford, CT (US); David J. Stone, Seattle, WA (US); Robert W. Gerwien, Meriden, CT (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/810,849

(22) Filed: Mar. 26, 2004

Related U.S. Application Data
(60) Provisional application No. 60/457,753, filed on Mar. 26, 2003.

(51) Int. Cl. C12Q 1/68

(52) U.S. Cl. 435/6

(57) ABSTRACT

Disclosed are methods of identifying psychoactive agents, e.g., anti-depressant, anti-psychotic or opioid compound, using differential gene expression.

### RELATED APPLICATIONS

This application claims priority to U.S. Ser. No. 60/457,753 filed Mar. 26, 2003, which is hereby incorporated by reference herein in its entirety

### FIELD OF THE INVENTION

The invention relates generally to the identification of genetic markers associated with psychoactive compounds.

### BACKGROUND OF THE INVENTION

Microarray-based gene expression patterns can be used as fingerprints of cellular physiology. The variety of cellular physiologies discernable by gene expression profile fingerprinting is expanding as an increasing range of cell types and cellular manipulations are investigated, and statistical methods of expression profile classification are refined. In yeast, distinctive profiles of genomic expression have been used to characterize cellular responses to diverse environmental transitions, functionally classify genetic manipulations and discover a novel target for a drug of partially characterized function. In cancer studies, microarray data has been used to classify solid tumors, correlate tumor characteristics to clinical outcome, and cluster cell lines on the basis of their tissue of origin and response to drugs In the area of toxicogenomics, large-scale analysis of toxin-treated cells and animals has resulted in a highly accurate capacity to recognize toxic profiles induced by novel drug candidates on the basis of gene expression, resulting in an increase in the efficiency of drug triage in the pharmaceutical development pipeline.

### SUMMARY OF THE INVENTION

The invention is based in part on the discovery that certain nucleic acids are differentially expressed neuronal cells treated with psychoactive compounds. Psycoactive compounds include for example, an antidepressant compound, an antipsychotic compound or an opioid receptor agonist These differentially expressed nucleic acids while previously described, have not heretofore been identified as associated with psychoactive activity and are collectively referred to herein as “PSYCHMARKER nucleic acids” or “PSYCHMARKER polynucleotides” and the corresponding encoded polypeptides are referred to as “PSYCHMARKER polypeptides” or “PSYCHMARKER proteins”. The PSYCHMARKER genes are useful in high throughput screening of potential therapeutic compounds for psychoactive activity. By psychoactive avtivity is meant that the compound alleviates a sign or symptom of a psychiatric disorder of depression, schizophrenia or pain.

In one aspect the invention provides methods of identifying a psychoactive compound or identifying drug efficacy of a psychoactive compound. By drug efficacy is meant that the compound confers a clinical benefit such as alleviating a symptom of a psychiatric disorder of depression, schizophrenia or pain. Pyschoactive compounds are identified or drug efficacy is determinedby determining the level of expression of a psychoactive-associated gene in a cell exposed to a test agent. The level of expression of the psychoactive-associated gene is compared to the level of expression of the psychoactive-associated gene in a control population exposed to a control agent. A test agent is predicted to have psychoactive activity if an alteration (e.g., increase or decrease) in the level of expression in the cell exposed to the test agent compared to the control population is identified. The method further provides for the identification of a functional category (e.g., antidepressant, antipsychotic compound or opioid receptor agonist) of a psychoactive drug to be determined.

Also provided by the invention are methods of screening a test agent for inducing changes in gene expression associated with a psychoactive compound. An agent is screened for inducing changes in gene expression associated with a psychoactive compound by determining the level of expression of a psychoactive-associated gene in a cell exposed to a test agent. The level of expression of the psychoactive-associated gene is compared to the level of expression of the psychoactive-associated gene in a control population exposed to a control agent.

The alteration is statistically significant. By statistically significant is meant that the alteration is greater than what might be expected to happen by change alone. Statistical significance is determined by method known in the art. An alteration is statistically significant if the p-value is at least 0.05. Preferably, the p-value is 0.04, 0.03, 0.02, 0.01, 0.005, 0.001 or less.

By psychoactive-associated gene is meant a gene that is characterized by a level of expression which differs in a cell exposed to a psychoactive compound compared to a control population. A psychoactive-associated gene includes for example PSYCHMARKER 1-13.

A control population is a for example a cell not exposed to a psychoactive compound. Optionally, the control population is exposed to a control agent. A control agent is an agent that does not elicit psychoactive activity. A control level is a single expression pattern derived from a single control population or from a plurality of expression patterns. For example, the control level can be a database of expression patterns from previously tested cells.

The test cell is provided in vitro. Alternatively, the test cell is provided ex vivo or in vivo from a mammalian subject. The test cell is derived from neuronal tissue. Expression is determined by for example detecting hybridization, e.g., on a chip, of a toxicity-associated gene probe to a gene transcript of the test cell.

The invention also provides a psychoactive compound reference expression profile of a gene expression level two or more of PSYCHMARKER 1-13. For example, the reference profile contains the expression levels of PSYCHMARKER 1-13.

The invention also provides a kit with a detection reagent which binds to two or more PSYCHMARKER nucleic acid sequences or which binds to a gene product encoded by the nucleic acid sequences. Also provided is an array of nucleic acids, e.g. oligonucleotides that binds to two or more PSYCHMARKER nucleic acids. For example, the array contains oligonucleotides that bind PSYCHMARKER 1-13.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION

The present invention is based in part on the discovery of changes in expression patterns of multiple nucleic acid sequences in response to compounds known to elicit psychoactive activity, e.g., antidepressant activity, antipsychotic activity or opioid receptor agonist activity. The differences in gene expression were identified by treating human primary neurons with multiple members of multiple classes of antidepressant drugs, antipsychotic drugs and opioid receptor agonists, followed by DNA microarray analysis of gene expression, to derive example gene expression profiles from these drug treatments. These expression profiles were used to construct statistical models capable of predicting drug efficacy based on a gene expression pattern. The set of biomarker genes that were derived, when considered together, predict the functional category of members of each of these drug classes (e.g., antidepressant, antipsychotic, or opioid receptor agonist) with at least 83.3% (Random Forest) or 88.9% (Classification Tree) accuracy, based on analysis of their expression levels induced by a drug. The ability to identify drug efficacy or activity on the basis of expression of the mRNA of these genes induced in vitro is useful in validation of drug targets for the treatment of the psychiatric disorders of depression, schizophrenia and pain.

The genes whose expression levels are modulated (i.e., increased or decreased) response to a psychoactive compound are summarized in Tables 1 and are collectively referred to herein as“psychoactive compound-associated genes”, “PSYCHMARKER nucleic acids” or “PSYCHMARKER polynucleotides” and the corresponding encoded polypeptides are referred to as“PSYCHMARKER polypeptides” or “PSYCHMARKER proteins.” Unless indicated otherwise,“PSYCHMARKER ” is meant to refer to any of the sequences disclosed herein. (e.g., PSYCHMARKER 1-13). By measuring the expression of these genes in response to various agents, agents for treating psychiatric disorders of depression, schizophrenia and pain are identified.

TABLE 1
Psychoactive Compound-Associated Genes

| Gene | PSYCHMARKER |
|---|---|
| PTX3 pentaxin 3 | 1 |
| ILK integrin linked kinase | 2 |
| ENTPD6 Ectonucleoside triphosphate | 3 |
| diphosphohydrolase 6 | |
| GPCR CG50207 | 4 |
| SFRS7 splicing factor, arginine/serine-rich 7 | 5 |
| CBRC7TM_424 GPCR | 6 |
| APAFf-1apoptotic protease activating factor 1 | 7 |
| ERMAP erythroblast membrane-associated | 8 |
| protein | |
| CGFLC_31120 | 9 |
| LYPLA1 lysophospholipase I | 10 |
| LDHA Lactate dehydrogenase A | 11 |
| SCG3 | 12 |
| CG187232 | 13 |

The invention involves determining (e.g., measuring) the expression of at least one, and up to all the PSYCHMARKER genes listed in Table 1. Using sequence information provided the psychoactive compound-associated genes are detected and measured using techniques well known to one of ordinary skill in the art. For example, sequences within the sequence database entries corresponding to PSYCHMARKER sequences, are used to construct probes for detecting PSYCHMARKER RNA sequences in, e.g., northern blot hybridization analyses. As another example, the sequences can be used to construct primers for specifically amplifying the PSYCHMARKER sequences in, e.g, amplification-based detection methods such as reverse-transcription based polymerase chain reaction.

Expression level of one or more of the PSYCHMARKER sequences in the test cell population, e.g., a neuronal cell, is then compared to expression levels of the some sequences in a reference population. The reference cell population includes one or more cells for which the compared parameter is known. The compared parameter can be, e.g., psychoactive compound expression status. By “psychoactive compound expression status” is meant that it is known whether the reference cell has had contact with a psychoactive compound. If desired, a reference expression profile is generated. A reference profile is a single expression pattern derived from a single reference population or from a plurality of expression patterns. For example, the reference cell population can be a database of expression patterns from previously tested cells for which one of the herein-described parameters or conditions is known.

Whether or not comparison of the gene expression profile in the test cell population to the reference cell population reveals the presence, or degree, of the measured parameter depends on the composition of the reference cell population. For example, if the reference cell population is composed of cells not exposed to a known psychoactive compound, a similar gene expression level in the test cell population and reference cell population indicates the test compound is not a psychoactive compound. Conversely, if the reference cell population is made up of cells exposed to a psychoactive compound, a similar gene expression profile between the test cell population and the reference cell population indicates that the test compound is a psychoactive compound.

Expression of sequences in test and reference populations of cells are compared using any art recognized method for comparing expression of nucleic acid sequences. For example, expression can be compared using a gene microarray such as CuraChip™ CuraChip provides a high-throughput means of global mRNA expression analyses of cDNA sequences representing the Pharmaceutically Tractable Genome (PTG). The CuraChip™ cDNAs are represented as 30-mer oligodeoxyribonucleotides (oligos) on a glass microchip. Hybridization methods using the longer CuraChip™ oligos are more specific compared with methods using 25-mer oligos. CuraChip™ oligos are synthesized with a linker, purified to remove truncated oligos (which can influence hybridization strength and specificity), and spotted on a glass slide. Details of the method used are described in Example 1. Alternatively, expression can be compared using GENECALLING® methods as described in U.S. Pat. No. 5,871,697 and in Shimkets et al., Nat. Biotechnol. 17:798-803.

An PSYCHMARKER sequence in a test cell population can be considered altered in levels of expression if its expression level varies from the reference cell population by more than 1.0, 1.5, 2.0, 5.0, 10.0 or more fold from the expression level of the corresponding PSYCHMARKER sequence in the reference cell population

If desired, comparison of differentially expressed sequences between a test cell population and a reference cell population can be done with respect to a control nucleic acid whose expression is independent of the parameter or condition being measured. For example, a control nucleic acid is one which is known not to differ depending on the exposure of the cell to a psychoactive compound. Expression levels of the control nucleic acid in the test and reference nucleic acid can be used to normalize signal levels in the compared populations. Control genes can be, e.g,. β-actin, glyceraldehyde 3- phosphate dehydrogenase or ribosomal protein P1 (36B4).

The test cell population is compared to multiple reference cell populations. Each of the multiple reference populations may differ in the known parameter. Thus, a test cell population may be compared to a first reference cell population known to contain, e.g., cells exposed to an antidepressant compound, as well as a second reference population known to contain, e.g., an antipsychotic comound.

The test cell population can be divided into two or more subpopulations. The subpopulations can be created by dividing the first population of cells to create as identical a subpopulation as possible. This will be suitable, in, for example, in vitro or ex vivo screening methods. In some embodiments, various subpopulations can be exposed to a control agent, and/or a test agent, multiple test agents, or, e.g., varying dosages of one or multiple test agents administered together, or in various combinations.

The test cell population comprises a neuronal cell. Preferably, the test cell population is a human neuronal cell. Cells in the reference cell population are derived from a tissue type as similar to test cell. Alternatively, the control cell population is derived from a database of molecular information derived from cells for which the assayed parameter or condition is known.

The subject is preferably a mammal. The mammal can be, e.g., a human, non-human primate, mouse, rat, dog, cat, horse, or cow.

The expression of 1, 2, 3, 4, 5, 10 or more of the sequences represented by PSYCHMARKER 1-13 is determined and if desired, expression of these sequences can be determined along with other sequences whose level of expression is known to be altered according to one of the herein described parameters or conditions.

Expression of the genes disclosed herein is determined at the RNA level using any method known in the art. For example, Northern hybridization analysis using probes which specifically recognize one or more of these sequences can be used to determine gene expression. Alternatively, expression is measured using reverse-transcription-based PCR assays, e.g., using primers specific for the differentially expressed sequences.

Expression is also determined at the protein level, i.e., by measuring the levels of polypeptides encoded by the gene products described herein. Such methods are well known in the art and include, e.g., immunoassays based on antibodies to proteins encoded by the genes.

When alterations in gene expression are associated with gene amplification or deletion, sequence comparisons in test and reference populations can be made by comparing relative amounts of the examined DNA sequences in the test and reference cell populations.

### Prediction of Psychoactive Activity

In one aspect, the invention provides a method of predicting or identifying agents with psychoactive activity. By “psychoactive activity” is meant that the compound has antidepressant activity, antipsychotic activity or opioid receptor agonist activity. Psychoactive activity is measured by methods know to those skilled in the art. Antidepressant activity defined by the inhibition of monoamien oxidase, inhibition of serotonin reuptake, inhibition of norepinephrine reuptake, inhibition of dopamine reuptake or an increase in serotonergic neurotransmission. Antipsychotic activity is defined by dopamine, serotinin or glutamate receptor blockade or an allevaition of a symptom of a psychotoic disorders such as schizophrenia, biopolar disorder or mania. Opoid receptor agonist activity is defined the inhibition of an opiod receptor or the allevaition of pain.

The method is an in vivo method. Alternatively, the method is an in vitro method.

By predicting the psychoactive activity is meant that the test compound is more likely to be a psychoactive copmpound, i.e, an antidepressant, an antipsychotic or an opioid receptor agonist than not be psychoactive. Psychoactive activity is predicted by determining the level of expression of a psychoactive-associated gene in a cell exposed to a test agent. The level of expression of the psychoactive-associated gene is compared to the level of expression of the psychoactive-associated gene in a control population exposed to a control agent. A test agent is predicted to be psychoactive if an alteration (e.g., increase or decrease) in the level of expression in the cell exposed to the test agent compared to the control population is identified.

The toxicity-associated gene is for example PSYCHMARKER 1-13. The toxicity-associated gene is a nucleic acid sequences homologous to those listed in Table 1 as PSYCHMARKER 1-13. The sequences need not be identical to sequences including PSYCHMARKER 1-13, as long as the sequence is sufficiently similar that specific hybridization can be detected.

The cell population is contacted in vitro, or in vivo. Optionally, the cell population is contacted ex vivo with the agent or activated form of the agent.

Expression of the nucleic acid sequences in the test cell population is then compared to the expression of the nucleic acid sequences in a control population, which is a cell population that has not been exposed to the test agent, or, in some embodiments, a cell population exposed to the test agent. Comparison can be performed on test and reference samples measured concurrently or at temporally distinct times. An example of the latter is the use of compiled expression information, e.g., a sequence database, which assembles information about expression levels of known sequences following administration of various agents. For example, alteration of expression levels following administration of test agent can be compared to the expression changes observed in the nucleic acid sequences following administration of a control agent. Preferably, the alteration ioin expression levels are within the alteration range listed on Table 5. A control agent is a compound that is known to be psychoactive compound. Alternatively, the control agent is a compound that is not a psychoactive compound. Exemplary control compounds are listed in the Examples.

An alteration in expression of the nucleic acid sequence in the test cell population compared to the expression of the nucleic acid sequence in the control cell population that has not been exposed to the test agent indicates the test agent is a psychoactive compound.

The alteration is statistically significant. By statistically significant is meant that the alteration is greater than what might be expected to happen by change alone. Statistical significance is determined by method known in the art. Multiple statistical methods have been applied to classification and recognition of expression profiles. For example, supervised classification analysis methods that classify patterns of data based on prior knowledge of sample classes include linear discriminant analysis and genetic algorithm/K-nearest neighbors (See Toxicol. Sci. 2002, 67:232-240, FEBS Lett. 2002, 522: 24-28), Fisher discriminant analysis (See Bioinformatics, 2002, 18:1054-1063), support vector machines (See Proc. Natl. Acad. Sci. USA, 2002, 262-267), neural networks (See Cancer Res. 2002, 62:3493-3497) and tree-based analysis (Front. Biosci. 2002, 7:c62-c67). An alteration is statistically significant if the prediction is at least 80% accurate. Preferably, the prediction is at least 82%, 83%, 85%, 88%, 90%, 92%. 95%, 99% or more accurate. Alternatively, statistical significance is determined by p-value. The p-values is a measure of probability that a difference between groups during an experiment happened by chance. (P(Z≧Z_{observed})). For example, a p-value of 0.01 means that there is a 1 in 100 chance the result occurred by chance. The lower the p-value, the more likely it is that the difference between groups was caused by treatment. An alteration is statistically significant if the p-value is at least 0.05. Preferably, the p-value is 0.04, 0.03, 0.02, 0.01, 0.005, 0.001 or less.

The invention also includes a psychoactive compound identified according to this screening method. The differentially expressed PSYCHMARKER sequences identified herein also allow for the efficacy of a psychoactive compound to be determined or monitored. In this method, a test cell population from a subject is exposed to a test agent, ie. a. psychoactive compound. If desired, test cell populations can be taken from the subject at various time points before, during, or after exposure to the test agent. Expression of one or more of the PSYCHMARKER sequences, e.g., PSYCHMARKER: 1-13, in the cell population is then measured and compared to a control population which includes cells whose psychoactive compound expression status is known.

### Identifying Agents that Inhibit or Enhance a Psychoactive-Associated Gene

An agent that inhibits the expression or activity of psychoactive-associated gene is identified by contacting a test cell population expressing metastatic lesions of colorectal cancer associated upregulated gene with a test agent and determining the expression level of the psychoactive-associated gene.

The test cell population is any cell expressing the psychoactive-associated genes. For example, the test cell population contains a neuronal cell or is derived from a neuronal tissue. For example, the test cell is immortalized cell line derived from a neuronal tissue.

### Selecting a Therapeutic Agent for Treating Psychiatric Disorders of Depression, Schizophrenia and Pain that is Appropriate for a Particular Individual

Differences in the genetic makeup of individuals can result in differences in their relative abilities to metabolize various drugs. An agent that is metabolized in a subject to act as an psychoactive compound can manifest itself by inducing a change in gene expression pattern in the subject's cells. Accordingly, the differentially expressed PSYCHMARKER sequences disclosed herein allow for a putative therapeutic or prophylactic psychoactive compound to be tested in a test cell population from a selected subject in order to determine if the agent is a suitable psychoactive compound in the subject.

To identify a psychoactive compound, that is appropriate for a specific subject, a test cell population from the subject is exposed to a therapeutic agent, and the expression of one or more of PSYCHMARKER 1-13 sequences is determined.

The test cell population contains neuronal cells expressing psychoactive-associated gene. For example a test cell population is incubated in the presence of a candidate agent and the pattern of gene expression of the test sample is measured and compared to one or more reference profiles. An alteration in expression of one or more of the sequences PSYCHMARKER 1-13 in a test cell population relative to a reference cell population is indicative that the agent is therapeutic. An agent is therauputic if it confers a clinical benefit such as alleviating a sign or symptom of a depression disorder, a psychotic disorder or a pain-related disorder.

Symptom of depression include for example, persistently sad, anxious, or “empty” mood, feelings of hopelessness, pessimism, feelings of guilt, worthlessness, helplessness, loss of interest or pleasure in hobbies and activities that were once enjoyed, including sex, insomnia, early-morning awakening, or oversleeping, decreased appetite and/or weight loss, or overeating and weight gain, atigue, decreased energy, being slowed down, thoughts of death or suicide, suicide attempts, restlessness and irritability. Depression id diagnosed for example by a physian performing a complete history of the patient's symptoms including (1) when did the symptoms start (2) how long have they lasted (3) how severe are they and (4) have the symptoms occurred before, and, if so, were they treated and what treatment was received.

Psychotic disorder include schizphernia, schizoaffective disorder, brief psychotic disorder or mania. Symptoms of psychotic disorders include for example, delusions, hallucinations, disorganized speech (e.g., frequent derailment or incoherence) or grossly disorganized or catatonic behavior. Psychotic disorders are diagnosed by a doctor performing a complete medical history and physical examination to determine the cause of the symptoms. No laboratory tests to specifically diagnose psychotic disorders—except those that accompany a physical illness, such as a brain tumor—the doctor may use various tests, such as blood tests and X-rays, to rule out physical illness as the cause of the symptoms.

### Kits

The invention also includes a PSYCHMARKER-detection reagent, e.g., a nucleic acid that specifically binds to or identifies one or more PSYCHMARKER nucleic acids such as oligonucleotide sequences, which are complementary to a portion of a PSYCHMARKER nucleic acid or antibodies which bind to proteins encoded by a PSYCHMARKER nucleic acid. For example the olignucleotides are 200, 150, 100, 50, 25, 10 or less nucleotides in length. The reagents are packaged together in the form of a kit. The reagents are packaged in separate containers, e.g., a nucleic acid or antibody (either bound to a solid matrix or packaged separately with reagents for binding them to the matrix), a control reagent (positive and/or negative), and/or a detectable label. Instructions (e.g., written, tape, VCR, CD-ROM, etc.) for carrying out the assay are included in the kit. The assay format of the kit is a Northern hybridization or a sandwich ELISA known in the art.

For example, PSYCHMARKER detection reagent, is immobilized on a solid matrix such as a porous strip to form at least one PSYCHMARKER detection site. The measurement or detection region of the porous strip may include a plurality of sites containing a nucleic acid. A test strip may also contain sites for negative and/or positive controls. Alternatively, control sites are located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of PSYCHMARKER present in the sample. The detection sites may be configured in any suitably detectable shape and are typically in the shape of a bar or dot spanning the width of a teststrip.

Alternatively, the kit contains a nucleic acid substrate array comprising one or more nucleic acid sequences. The nucleic acids on the array specifically identify one or more nucleic acid sequences represented by PSYCHMARKER 1-13. The expression of 2, 3, 4, 5, 6, 7, 8, 9, 10, or more of the sequences represented by PSYCHMARKER 1-13 are identified by virtue if the level of binding to an array test strip or chip. The substrate array can be on, e.g., a solid substrate, e.g., a “chip” as described in U.S. Pat. No. 5,744,305.

### Arrays and Pluralities

The invention also includes a nucleic acid substrate array comprising one or more nucleic acid sequences. The nucleic acids on the array specifically corresponds to one or more nucleic acid sequences represented by PSYCHMARKER 1-13. The level expression of 2, 3, 4, 5, 6, 7, 8, 9, 10, or more of the sequences represented by PSYCHMARKER 1-13 are identified by detecting nucleic acid binding to the array.

The invention also includes an isolated plurality (i.e., a mixture if two or more nucleic acids) of nucleic acid sequences. The nucleic acid sequence are in a liquid phase or a solid phase, e.g., immobilized on a solid support such as a nitrocellulose membrane. The plurality includes one or more of the nucleic acid sequences represented by PSYCHMARKER 1-13. In various embodiments, the plurality includes 2, 3, 4, 5, 6, 7, 8, 9, 10, or more of the sequences represented by PSYCHMARKER 1-13.

### Chips

The DNA chip is a device that is convenient to compare expression levels of a number of genes at the same time. DNA chip-based expression profiling can be carried out, for example, by the method as disclosed in “Microarray Biochip Technology ” (Mark Schena, Eaton Publishing, 2000), etc.

A DNA chip comprises immobilized high-density probes to detect a number of genes. Thus, expression levels of many genes can be estimated at the same time by a single-round analysis. Namely, the expression profile of a specimen can be determined with a DNA chip. The DNA chip-based method of the present invention comprises the following steps of:

(1) synthesizing cRNAs or cDNAs corresponding to the marker genes;

(2) hybridizing the cRNAs or cDNAs with probes for marker genes; and

(3) detecting the cRNA or cDNA hybridizing with the probes and quantifying the amount of mRNA thereof.

The cRNA refers to RNA transcribed from a template cDNA with RNA polymerase. A cRNA transcription kit for DNA chip-based expression profiling is commercially available. With such a kit, cRNA can be synthesized from T7 promoter-attached cDNA as a template by using T7 RNA polymerase. On the other hand, by PCR using random primer, cDNA can be amplified using as a template a cDNA synthesized from mRNA.

On the other hand, the DNA chip comprises probes, which have been spotted thereon, to detect the marker genes of the present invention. There is no limitation on the number of marker genes spotted on the DNA chip. For example, it is allowed to select 5% or more, preferably 20% or more, more preferably 50% or more, still more preferably 70% or more of the marker genes of the present invention. Any other genes as well as the marker genes can be spotted on the DNA chip. For example, a probe for a gene whose expression level is hardly altered may be spotted on the DNA chip. Such a gene can be used to normalize assay results when assay results are intended to be compared between multiple chips or between different assays.

A probe is designed for each marker gene selected, and spotted on a DNA chip. Such a probe may be, for example, an oligonucleotide comprising 5-50 nucleotide residues. A method for synthesizing such oligonucleotides on a DNA chip is known to those skilled in the art. Longer DNAs can be synthesized by PCR or chemically. A method for spotting long DNA, which is synthesized by PCR or the like, onto a glass slide is also known to those skilled in the art. The prepared DNA chip is contacted with cRNA, followed by the detection of hybridization between the probe and cRNA. The cRNA can be previously labeled with a fluorescent dye. A fluorescent dye such as Cy3(red) and Cy5 (blue) can be used to label a cRNA. cRNAs from a subject and a control are labeled with different fluorescent dyes, respectively. The difference in the expression level between the two can be estimated based on a difference in the signal intensity. The signal of fluorescent dye on the DNA chip can be detected by a scanner and analyzed by using a special program. For example, the Suite from Affymetrix is a software package for DNA chip analysis.

Also the expression level of the marker gene(s) can be analyzed based on activity or quantity of protein(s) encoded by the marker gene(s). A method for determining the quantity of the protein(s) is known to those skilled in the art. For example, immunoasssay method is useful for determination of the protein in biological material. Any biological materials can be used for the determination of the protein or it's activity. Alternatively, a suitable method is selected for the determination of the activity protein(s) encoded by the marker gene(s) according to the activity of each protein to be analyzed.

### EXAMPLE 1

### General Methods

To generate expression profiles of different drug class treatments, antidepressants antipsychotics and opioid receptor agonists were administerd to primary human neuronal cultures. The assays were carried out as follows.

Cell Culture

Primary human neuronal precursor cells (Clonexpress, Gaithersberg, Md.) derived from 14-16 week embryos were cultured seven days in growth media (50:50 DMEM/F12, 5% FBS, 10 ng/ml bFGF, 10 ng/ml EGF, 1:100 Clonexpress neuronal cell supplement (NCS), pen/strep). To differentiate into neuronal phenotype, cells were plated in poly-l-lysine coated 6-well plates at 900,000 cells/well and cultured seven days in differentiation media (50:50 DMEM/F12+5% FBS, 10 ng/ml bFGF, 10 ng/ml EGF, 1:100 NCS, pen/strep, 100 μM dibutyrtyl CAMP, 20 ng/ml NGF, 1:100 matrigel), with 72 hr media changes. Morphologically neuronal cells comprised approximately 80% of the cultures.

Drug Treatments

To generate expression profiles of different drug class treatments, twenty antidepressants (five selective serotonin reuptake inhibitor, four serotonin receptor agonists, seven monoamine oxidase inhibitor, three norepinephrine uptake inhibitors), eight antipsychotics (four classic neuroleptics, four atypical antipsychotics) and eight opioid receptor agonists (two delta receptor agonists, three mu receptor agonists, three kappa receptor agonists) were administerd to primary human neuronal cultures. Drugs used for treatment were dissolved in DMSO and added to cultures to achieve a final DMSO concentration of 0.04%. Final drug concentrations represented pharmaceutically relevent doses: amoxepine, 2.0 μM; clomipramine, 2.0 μM; desipramine, 2,0 μM; doxepin, 1.0 μM; imipramine, 2.0 μM; maprotiline, 1.0 1 M; nortryptyline, 0.7 μM; protriptyline, 0.4 μM; trimipramine, 1.5 μM; citalopram, 0.3 μM; paroxetine, 0.3 μM; sertraline, 1.4 μM; tranylcypromine, 0.4 μM; phenylzine, 0.8 μM; iproniazid, 0.6 μM; trazadone, 2.0 μM; amitriptyline, 1.0 μM; fluoxetine, 0.5 μM; fluvoxamine, 1.5 μM; bupropion, 2.3 μM; chlorpromazine, 1.0 μM; trifluperazine, 1.0 μM; triflupromazine, 0.8 μM; pimozide, 0.05 μM; clozapine, 4.0 μM, haloperidol, 0.2 μM; risperidone, 0.04 μM; loxapine, 0.5 μM; BW373U86, 0.1 nM; Enkephalin, 1.0 μM; U50488, 0.1 nM; U62066, 1.0 μM; Endomorphin, 1.0 μM; DALDA, 0.1; DAMGO, 0.1 μM; Dynorphin A, 0.1 μM. All treatments were 24 hrs in duration and were conducted simultaneously. All drugs were purchased from Sigma-Aldrich (St. Louis, Mo.) or Tocris Cookson (Ellisville, Mo.).

Sample Processing

Drug-treated cell cultures were lysed in Trizol. Total RNA was extracted by phenol chloroform extraction. Each sample evaluated for gene expression by hybridization to the proprietary CuraChip microarray (CuraGen, New Haven, Conn.) of approximately 11,000 oligonucleotide probes corresponding to the component of the human genome encoding proteins with activities in classes targetable by pharmaceutical means (the “pharmaceutically tractable genome”). Biotin-labeled cDNA was made using 15 ug of total RNA using poly-T oligo primers. Slides were hybridized at 30° C. for 15 hours with constant rotation, then washed for 30 min at RT. Slides were incubated in streptavidin solution (4° C., 30 min) and washed 3× for 15 min at RT, followed by incubation in Cy3-conjugated detection buffer (4° C., 30 min) and again washed 3× for 15 min at RT. Slides were then scanned (GMS 418 Scanner, Genetic Microsystems, Woburn, Mass.) and images analyzed using Imagene software (BioDiscovery, Marina Del Rey, Calif.). Of the 11,000 genes on the microarray, approximately 4,700 were found to be expressed by primary human neuronal cultures at three-fold above background.

### EXAMPLE 2

### Identification of Genes Associated with Psychoactive Compounds

All genes detectable at three-fold over background after signal normalization were included in the data sets for analysis. Data was pre-filtered using a generous Kruskal-Wallis filter (p<0.001, ˜4700 genes over 36 samples). Classification Trees were calculated within a leave one out cross-validation loop to minimize the influence of marker pre-filtering on model accuracy. The markers selected for the decision tree were then removed from the data set and this process was repeated two additional times. For each iteration of the classification tree algorithm, the samples were weighted such that an unknown would have an equal probability of falling within either class, and not default to the over represented class (antidepressants). The Random Forest algorithm was also calculated within a leave one out cross-validation loop using pre-filtered data, with 1000 trees grown and 2 random inputs attempted at each split. All statistical algorithms were performed using the ‘R’ statistical software system (http://www.cran.r-project.org/). The result of this process is the identification of a set of biomarker genes for the defined drug classes. (Table 4)

Supervised Classification of Drug-Treated Samples

The supervised classification methods of Random Forest and Classification Tree were used to analyze the gene expression profiles of drug-treated neurons, as has been described previously¹⁹. These methods have the advantage that examples of known classes can be used to build models of salient features that provide categorical distinction between the data sets used to build the models. These models can be tested empirically with data sets of known class that were not used in model construction (cross validation). With both the Classification Tree and Random Forest methods, a “leave one out” training and testing series was conducted for all 36 drug treated samples. Thus, 36 individual models were constructed, each trained with 35 example gene expression profiles, with one profile withheld from training for evaluation. After construction of each model, the profile excluded from the training set was tested by the model for assignment to one of the three drug treatment categories (antidepressant, antipsychotic or opioid receptor agonist). The overall effectiveness of each method was calculated as the percent correct classifications out of the total 36 training and testing events conducted by the method.

Classification Tree

The Classification Tree method classified 32 out of 36 expression profiles in the category corresponding to the therapeutic application of the drug used to treat the cells (88.9% correct classification) (Table. 2). Of the 20 antidepressants, 18 were correctly classified, one was classified as an antipsychotic and one was classified as an opioid receptor agonist. Of the eight antipsychotics, seven were correctly classified and one was classified as an antidepressant. Of the eight opioid receptor agonists, seven were correctly classified and one was classified as an antidepressant. Interestingly, as few as four gene markers were sufficient to provide this level of resolution accuracy among these expression profiles. The identity of these genes is: PTX3 (pentaxin 3), ILK (integrin linked kinase), ENTPD6 (ectonucleoside triphosphate diphosphohydrolase 6), and a novel G protein-coupled receptor, GPCR CG50207. PTX3 (pentaxin 3) and ILK (integrin linked kinase) were sufficient to provide the majority of resolution between classes, as indicated by the high marker count for these genes (Table 3). Three-dimensional graphical representation of all possible three-way combinations of these four biomarkers illustrates the robust class separation provided by expression level comparison

[image]

TABLE 3
Marker sets resulting from three sequential iterations of the
Classification Tree analysis method

| | |
|---|---|
| Marker identity | Marker count |

Iteration 1

| | |
|---|---|
| PTX3 pentaxin 3 | 35 |
| ILK integrin linked kinase | 34 |
| ENTPD6 Ectonucleoside triphosphate | 1 |
| diphosphohydrolase 6 | |
| GPCR CG50207 | 1 |

Iteration 2

| | |
|---|---|
| SFRS7 splicing factor, arginine/serine-rich 7 | 34 |
| ENTPD6 Ectonucleoside triphosphate | 24 |
| diphosphohydrolase 6 | |
| CBRC7TM_424 GPCR | 1 |
| APAFf-1apoptotic protease activating factor 1 | 1 |
| ERMAP erythroblast membrane-associated protein | 8 |
| CGFLC_31120 | 1 |
| GPCR CG50207 | 1 |
| LDHA Lactate dehydrogenase A | 1 |

Iteration 3

| | |
|---|---|
| LYPLA1 lysophospholipase 1 | 34 |
| GPCR CG50207 | 26 |
| CBRC7TM_424 GPCR | 1 |
| APAFf-1apoptotic protease activating factor 1 | 8 |
| CGFLC_31120 | 1 |
| LDHA Lactate dehydrogenase A | 1 |

For each iteration, the two most frequent markers from the previous iteration were deleted from the expression data set.

Marker identities are given where possible; GPCR CG50207 and CGFLC_{—}31120 are novel sequences of GPCR and unknown function, respectively. Marker count represents the number of times out of the 36 model building episodes a particular gene was selected as a marker.

Random Forest

The Random Forest method classified 30 out of 36 expression profiles in the category corresponding to the therapeutic application of the drug used to treat the cells (83.3% correct classification) (Table 2). Of the 20 antidepressants, 18 were correctly classified and two were classified as antipsychotics. Of the eight antipsychotics, seven were correctly classified and one was classified as antidepressant. Of the eight opioid receptor agonists, five were correctly classified and three were classified as antidepressants. The Random Forest analysis identified 326 markers that were used to construct the predictive models. The markers assumed an importance measure between zero and one, relating to the strength of their respective contributions to the models. A large importance measure indicates that random permutation of that gene causes samples to be misclassified more often (hence that gene is important). 32 of the markers had an importance measure greater than 0.35. Three had an importance measure above 0.75: SFRS7 (splicing factor, arginine/serine-rich 7), SCG3 (secretogranin III), and hypothetical protein CG187232-01. The class separation provided by only these three top biomarkers is less distinct than the separation yielded by the biomarkers identified by the Classification Tree. This is probably due to the relative proficiency of the Classification Tree and Random Forest algorithms with data sets containing a few strong markers, or a large number of weak markers, respectively.

Multiple Iterations of Classification Tree

Since relatively few genes were identified as drug class markers by the Classification Tree, we investigated the robustness of the approach in the absence of these markers by successively performing a second and third iteration of the analysis, excluding from the data set the predominant gene markers identified by the first and second iterations, respectively (Table 4). The second iteration resulted in the identification of eight gene markers sufficient to provide 72.2% resolution accuracy. Interestingly, the third iteration resulted in the identification of fewer gene markers (six) than the second iteration, sufficient to provide greater resolution accuracy (80.6%) between treatment classes, even though the strongest markers from the second iteration were removed from the data set.

[image]

[image]

Table 5 summarizes the expression level range in the form of relative fluorescence units (RFU), for each biomarker required for effective categorization to occur.

TABLE 5

| Genes | Max AD | Min AD | Max AP | Min AP | Max OP | Min OP |
|---|---|---|---|---|---|---|
| PTX3 pentaxin 3 | 1.081475 | 0.688761 | 1.036963 | 0.601615 | 1.696653 | 1.086105 |
| ILK integrin linked kinase | 1.276744 | 0.764877 | 0.915803 | 0.739284 | 1.175496 | 0.858477 |
| ENTPD6 Ectonucleoside | 1.549311 | 0.786736 | 1.076364 | 0.573707 | 1.477081 | 0.837467 |
| triphosphate diphosphohydrolase 6 | | | | | | |
| GPCR CG50207 | 1.826622 | 0.852346 | 1.321024 | 0.493227 | 1.464915 | 0.339308 |
| SFRS7 splicing factor, | 1.12308 | 0.5331 | 1.0783 | 0.687398 | 1.359178 | 1.123489 |
| arginine/serine-rich 7 | | | | | | |
| CBRC7TM_424 GPCR | 1.600307 | 0.067351 | 2.401118 | 1.036077 | 0.938608 | 0.497713 |
| APAFf-1 apoptotic protease | 1.680057 | 0.696118 | 0.905628 | 0.606785 | 1.299162 | 0.81503 |
| activating factor 1 | | | | | | |
| ERMAP erythroblast membrane- | 1.241793 | 0.449553 | 1.591711 | 1.194523 | 1.28326 | 0.427542 |
| associated protein | | | | | | |
| CGFLC_31120 | 1.152633 | 0.659439 | 1.750288 | 0.908919 | 1.45432 | 0.664492 |
| LYPLA1 lysophospholipase I | 1.195241 | 0.394012 | 1.274685 | 0.672949 | 1.986995 | 1.328546 |
| LDHA Lactate dehydrogenase A | 1.182659 | 0.420308 | 1.218164 | 0.588735 | 1.725345 | 1.116754 |
| SCG3 | 1.198662 | 0.343028 | 0.984209 | 0.602056 | 1.482699 | 1.179987 |
| CG187232 | 1.401032 | 0.338927 | 0.91906 | 0.369117 | 2.340879 | 0.824419 |

### EXAMPLE 3

### Psychoactive-Associated Genes

This example provides exemplary PSYCHMARKER nucleic acid sequences, useful in methods of screening compounds for psychoactive activity according to the invention

PTX3 pentaxin 3 (PSYCHMARKER 1; SEQ ID NO :1)
ATGCATCTCC TTGCGATTCT GTTTTGTGCT CTCTGGTCTG CAGTGTTGGC CGAGAACTCG
GATGATTATG ATCTCATGTA TGTGAATTTG GACAACGAAA TAGACAATGG ACTCCATCCC
ACTGAGGACC CCACGCCGTG CGACTGCGGT CAGGAGCACT CGGAATGGGA CAAGCTCTTC
ATCATGCTGG AGAACTCGCA GATGAGAGAG CGCATGCTGC TGCAAGCCAC GGACGACGTC
CTGCGGGGCG AGCTGCAGAG GCTGCGGGAG GAGCTGGGCC GGCTCGCGGA AAGCCTGGCG
AGGCCGTGCG CGCCGGGGGC TCCCGCAGAG GCCAGGCTGA CCAGTGCTCT GGACGAGCTG
CTGCAGGCGA CCCGCGACGC GGGCCGCAGG CTGGCGCGTA TGGAGGGCGC GGAGGCGCAG
CGCCCAGAGG AGGCGGGGCG CGCCCTGGCC GCGGTGCTAG AGGAGCTGCG GCAGACGCGA
GCCGACCTGC ACGCGGTGCA GGGCTGGGCT GCCCGGAGCT GGCTGCCGGC AGGTTGTGAA
ACAGCTATTT TATTCCCAAT GCGTTCCAAG AAGATTTTTG GAAGCGTGCA TCCAGTGAGA
CCAATGAGGC TTGAGTCTTT TAGTGCCTGC ATTTGGGTCA AAGCCACAGA TGTATTAAAC
AAAACCATCC TGTTTTCCTA TGGCACAAAG AGGAATCCAT ATGAAATCCA GCTGTATCTC
AGCTACCAAT CCATAGTGTT TGTGGTGGGT GGAGAGGAGA ACAAACTGGT TGCTGAAGCC
ATGGTTTCCC TGGGAAGGTG GACCCACCTG TGCGGCACCT GGAATTCAGA GGAAGGGCTC
ACATCCTTGT GGGTAAATGG TGAACTGGCG GCTACCACTG TTGAGATGGC CACAGGTCAC
ATTGTTCCTG AGGGAGGAAT CCTGCAGATT GGCCAAGAAA AGAATGGCTG CTGTGTGGGT
GGTGGCTTTG ATGAAACATT AGCCTTCTCT GGGAGACTCA CAGGCTTCAA TATCTGGGAT
AGTGTTCTTA GCAATGAAGA GATAAGAGAG ACCGGAGGAG CAGAGTCTTG TCACATCCGG
GGGAATATTG TTGGGTGGGG AGTCACAGAG ATCCAGCCAC ATGGAGGAGC TCAGTATGTT
TCATAA
ILK integrin linked kinase (PSYCHMARKER 2; SEQ ID NO :2)
TTTTTTTTTTTTTTTTTTTTTTCATAATAAACTTTATTGTGACAGGCGGGGCTGATCCCT
CCCATGTTGGAAGACACCATGTGGCAAGTGACAAAGCTCTGAGCCCGCCCCTCTTGCGCA
CAGTGGTAGGGATGGGGGAAGGGGATGGACCCCAGGCTGGGGTAGTACCATGACTGGAGG
CGGGGGAGGCAACCAGAGGCCTGCTGCTTTGGGGAGGTGCATTCCCCCAACCATGTCCCG
ACACCTCTGGAGTTCAGGCAAGGACCTTCCAGTCCTACTTGTCCTGCATCTTCTCAAGGA
TAGGCACAATCATGTCAAATTTGGGTCGCTTTGCAGGGTCTTCATTCATGCAGATCTTCA
TGAGCTTACACACATGAGGGGAAATACCTGGTGGGATGGTAGGCCGAAGGCCTTCCAATG
CCACCTTCATTCCAATCTCCATATTGGAGAGGTCAGCAAAGGGTACCTCCCGTGTCACCA
GTTCCCACAGAAGCACTGCAAAACTCCACATGTCTGCTGAGCGTCTGTTTGTGTCTTCAG
GCTTCTTCTGCAGAGCTTCGGGGGCTACCCAGGCAGGTGCATACATGCGACCAGGACATT
GGAAAGAGAACTTGACATCAGCCATGCTAATTCGGGCAGTCATGTCCTCATCAATCATTA
CACTACGGCTATTGAGTGCATGTCGTGGGATGAGGGGCTCTAGTGTGTGTAGGAAGGCCA
TGCCCCTTGCCATGTCCAAAGCAAACTTCACAGCCTGGCTCTGGTCCACGACGAAATTGG
TGCCTTCATGTAGTACATTGTAGAGGGATCCATACGGCATCCAGTGTGTGATGAGAGTAG
GATGAGGAGCAGGTGGAGACTGGCAGGCACCTAGCACTGGGAGCACATTTGGATGCGAGA
AAATCCTGAGCCGGGGACACTCTTCATTGAAGTCCCTGCTCTTCCTTGTACTCCAGTCTC
GAACCTTCAGCACCTTCACGACAATGTCATTGCCCTGCCAGCGGCCCTTCCATAGCTCTC
CAGAGTGATTCTCGTTGAGCTTCGTCAGGAAGTTAAGCTGTTTGAAGTCAATGCCAGAGT
GTTTGTTCAGGGTTCCATTTCGGGGCCGAGTGCGGGTGGTCCCCTTCCAGAATGTGTCCT
TGTATGGAATACGGTTGAGATTCTGGCCCATCTTCTCTGCCCGCTCTCGGAGAAGCTCTC
TCAGGGGTGCCTTGGCTTTGTCCACAGGCATCTCTCCATACTTGTTACAGATGCTGACAA
GGGCCCCATTTGCCACCAGGTCCTCTGCCACTTGATCTTGGCCCCAAAAACAGGCATAGT
GCAGGGGCACATTCCCGTGTTCATTCACTGCATTGATGTCTGCCTTGTACTGCAATAGCT
TCTGTACAATATCACGGTGTCCATGACTGGCTGCCAGATGCAGGGGGGTGTCATCCCCAC
GGTTCATTACATTGATCCGTGCCCCCCGCATGATCAACATCTCAACCACAGCAGAGCGGC
CCTCTCGGCAGGCCCAGTGCAAGGGGGAGAAGCCATGATCGTCCCCCTGGTTGAGGTCGT
TCTCCGTGTTGTCCAGCCACAGGCGAACGGCGACTGCGTTGCCCTCCCGGCACTGAGTGA
AAATGTCGTCCATAGCAGCGTCCCGGCGCCGAGTCCCCTGGATTGGGGAAGCCTGAGGAC
TGTGGAGTGATCCAGGGAAGGAGGATGAACCCCAAGCTTTATCCTCGGGACTCGGGCTGC
AGGATCCTTCTCCGGGGAACTCCCGTGGTAGCAGTCGACAGATGAATTC
ENTPD6 (Ectonucleoside triphosphate diphosphohydrolase 6)
(PSYCHMARKER 3; SEO ID NO :3)
CCCACCATGAAAAAAGGTATCCGTTATGAAACTTCCAGAAAAACGAACTACATTTTTCAG
CAGCCGCAGCACGGTCCTTGGCAAACAAGGATGAGAAAAATATCCAACCACGGGAGCCTG
CGGGTGGCGAAGGTGGCATACCCCCTGGGGCTGTGTGTGGGCGTGTTCATCTATGTTGCC
TACATCAAGTGGCACCGGGCCACCGCCACCCAGGCCTTCTTCAGCATCACCAGGGCAGCC
CCGGGGGCCCGGTGGGGTCAGCAGGCCCACAGCCCCCTGGGGACAGCTGCAGACGGGCAC
GAGGTCTTCTACGGGATCATGTTTGATGCAGGAAGCACTGGCACCCGAGTACACGTCTTC
CAGTTCACCCGGCCCCCCAGAGAAACTCCCACGTTAACCCACGAAACCTTCAAAGCACTG
AAGCCAGGTCTTTCTGCCTATGCTGATGATGTTGAAAAGAGCGCTCAGGGAATCCGGGAA
CTACTGGATGTTGCTAAACAGGACATTCCATTCGACTTCTGGAAGGCCACCCCTCTGGTC
CTCAAGGCCACAGCTGGCTTACGCCTGTTACCTGGAGAAAAGGCCCAGAAGTTACTGCAG
AAGGTGAAAGAAGTATTTAAAGCATCGCCTTTCCTTGTAGGGGATGACTGTGTTTCCATC
ATGAACGGAACAGATGAAGGCGTTTCGGCGTGGATCACCATCAACTTCCTGACAGGCAGC
TTGAAAACTCCAGGAGGGAGCAGCGTGGGCATGCTGGACTTGGGCGGAGGATCCACTCAG
ATCGCCTTCCTGCCACGCGTGGAGGGCACCCTGCAGGCCTCCCCACCCGGCTACCTGACG
GCACTGCGGATGTTTAACAGGACCTACAAGCTCTATTCCTACAGCTACCTCGGGCTCGGG
CTGATGTCGGCACGCCTGGCGATCCTGGGCGGCGTGGAGGGGCAGCCTGCTAAGGATGGA
AAGGAGTTGGTCAGCCCTTGCTTGTCTCCCAGTTTCAAAGGAGAGTGGGAACACGCAGAA
GTCACGTACAGGGTTTCAGGGCAGAAAGCAGCGGCAAGCCTGCACGAGCTGTGTGCTGCC
AGAGTGTCAGAGGTCCTTCAAAACAGAGTGCACAGGACGGAGGAAGTGAAGCATGTGGAC
TTCTATGCTTTCTCCTACTATTACGACCTTGCAGCTGGTGTGGGCCTCATAGATGCGGAG
AAGGGAGGCAGCCTGGTGGTGGGGGACTTCGAGATCGCAGCCAAGTACGTGTGTCGGACC
CTGGAGACACAGCCGCAGAGCAGCCCCTTCTCATGCATGGACCTCACCTACGTCAGCCTG
CTACTCCAGGAGTTCGGCTTTCCCAGGAGCAAAGTGCTGAAGCTCACTCGGAAAATTGAC
AATGTTGAGACCAGCTGGGCTCTGGGGGCCATTTTTCATTACATCGACTCCCTGAACAGA
CAGAAGAGTCCAGCCTCA
CG50207 (GPCR) (PSYCHMARKER 4; SEQ ID NO :4)
ATGACGAACACATCATCCTCTGACTTCACCCTCCTGGGGCTTCTGGTGAACAGTGAGGCT
GCCGGGATTGTATTTACAGTGATCCTTGCTGTTTTCTTGGGGGCCGTGACTGCAAATTTG
GTCATGATATTCTTGATTCAGGTGGACTCTCGCCTCCACACCCCCATGTACTTTCTGCTC
AGTCAGCTGTCCATCATGGACACCCTTTTCATCTGTACCACTGTCCCAAAACTCCTGGCA
GACATGGTTTCTAAAGAGAAGATCATTTCCTTTGTGGCCTGTGGCATCCAGATCTTCCTC
TACCTGACCATGATTGGTTCTGAGTTCTTCCTCCTGGGCCTCATGGCCTATGACCGCTAC
GTGGCTGTCTGTAACCCTCTGAGATACCCAGTCCTGATGAACCGCAAGAAGTGTCTTTTG
CTGGCTGCTGGTGCCTGGTTTGGGGGCTCCCTCGATGGCTTTCTGCTCACTCCCATCACC
ATGAATGTCCCTTACTGTGGCTCCCGAAGTATCAACCATTTTTTCTGTGAGATCCCAGCA
GTTCTGAAACTGGCCTGTGCAGACACGTCCTTGTATGAAACTCTGATGTACATCTGCTGT
GTCCTCATGTTGCTCATCCCCATCTCTATCATCTCCACTTCCTACTCCCTCATCTTGTTA
ACCATCCACCGCATGCCCTCTGCTGAAGGTCGCAAAAAGGCCTTCACCACTTGTTCCTCC
CACTTGACTGTAGTTAGCATCTTCTATGGGGCTGCCTTCTACACATACGTGCTGCCCCAG
TCCTTCCACACCCCCGAGCAGGACAAAGTAGTGTCAGCCTTCTATACCATTGTCACGCCC
ATGCTTAATCCTCTCATCTACAGCCTCAGAAACAAGGACGTCATAGGGGCATTTAAAAAG
GTATTTGCATGTTGCTCATCTGCTCGGAAAGTAGCAACAAGTGATGCTTAGAGAGTCACT
GCCCAGAGGATAAGGCTTCCTAAGGACTTCCTC
SFRS7 (splicing factor, arginine/serine-rich 7)
(PSYCHMARKER 5; SEQ ID NO :5)
GTAGTGCCGCCGGGACTCTTGGCGGGTGAAGGTGTGTGTCAGCTTTTGCGTCACTCGAGC
CCTGGGCGCTGCTTGCTAAAGAGCCGAGCACGCGGGTCTGTCATCATGTCGCGTTACGGG
CGGTACGGAGGAGAAACCAAGGTGTATGTTGGTAACCTGGGAACTGGCGCTGGCAAAGGA
GAGTTAGAAAGGGCTTTCAGTTATTATGGTCCTTTAAGAACTGTATGGATTGCGAGAAAT
CCTCCAGGATTTGCCTTTGTGGAATTCGAAGATCCTAGAGATGCAGAAGATGCAGTACGA
GGACTGGATGGAAAGGTGATTTGTGGCTCCCGAGTGAGGGTTGAACTATCGACAGGCATG
CCTCGGAGATCACGTTTTGATAGACCACCTGCCCGACGTCCCTTTGATCCAAATGATAGA
TGCTATGAGTGTGGCGAAAAGGGACATTATGCTTATGATTGTCATCGTTACAGCCGGCGA
AGAAGAAGCAGGTCACGGTCTAGATCACATTCTCGATCCAGAGGAAGGCGATACTCTCGC
TCACGCAGCAGGAGCAGGGGACGAAGGTCAAGGTCAGCATCTCCTCGACGATCAAGATCT
ATCTCTCTTCGTAGATCAAGATCAGCTTCACTCAGAAGATCTAGGTCTGGTTCTATAAAA
GGATCGAGGTATTTCCAATCCCCGTCGAGGTCAAGATCAAGATCCAGGTCTATTTCACGA
CCAAGAAGCAGCCGATCAAAGTCCAGATCTCCATCTCCAAAAAGAAGTCGTTCCCCATCA
GGAAGTCCTCGCAGAAGTGCAAGTCCTGAAAGAATGGACTGAAGCTCTCAAGTTCACCCT
TTAGGGAAAAGTTATTTTGTTTACATTATTATAAGGGATTTGTGATGTCTGTAAAGTGTA
ACCTAGGAAAGATAATTCAACCATCTAATCAAAATGGATCTGGATTACTATGTAAATTCA
CAGCAGTAAGG
CBRC7TM 424 GPCR (PSYCHMARKER 6; SEQ ID NO :6)
ATGGATCAGA GAAATTACAC CAGAGTGAAA GAATTTACCT TCCTGGGAAT TACTCAGTCC
CGAGAACTGA GCCAGGTCTT ATTTACCTTC CTGTTTTTGG TGTACATGAC AACTCTAATG
GGAAACTTCC TCATCATGGT TACAGTTACC TGTGAATCTC ACCTTCATAC GCCCATGTAC
TTCCTGCTCC GCAACCTGTC TATTCTTGAC ATCTGCTTTT CCTCCATCAC AGCTCCTAAG
GTCCTGATAG ATCTTCTATC AGAGACAAAA ACCATCTCCT TCAGTGGCTG TGTCACTCAA
ATGTTCTTCT TCCACCTTCT GGGGGGAGCA GACGTTTTTT CTCTCTCTGT GATGGCGTTT
GACCGCTATA TAGCCATCTC CAAGCCCCTG CACTATATGA CCATCATGAG TAGGGGGCGA
TGCACAGGCC TCATCGTGGC TTCCTGGGTG GGGGGCTTTG TCCACTCCAT AGCGCAGATT
TCTCTATTGC TCCCACTCCC TTTCTGTGGA CCCAATGTTC TTGACACTTT CTACTGCGAT
GTCCCCCAGG TCCTCAAACT TGCCTGCACT GACACCTTCA CTCTGGAGCT CCTGATGATT
TCAAATAATG GGTTAGTCAG TTGGTTTGTA TTCTTCTTTC TCCTCATATC TTACACGGTC
ATCTTGATGA TGCTGAGGTC TCACACTGGG GAAGGCAGGA GGAAAGCCAT CTCCACCTGC
ACCTCCCACA TCACCGTGGT GACCCTGCAT TTCGTGCCCT GCATCTATGT CTATGCCCGG
CCCTTCACTG CCCTCCCCAC AGACACTGCC ATCTCTGTCA CCTTCACTGT CATCTCCCCT
TTGCTCAATC CTATAATTTA CACGCTGAGG AATCAGGAAA TGAAGTTGGC CATGAGGAAA
CTGAAGAGAC GGCTAGGACA ATCAGAAAGG ATTTTAATTC AATAA
APAF-1 (apoptotic protease activating factor 1)
(PSYCHMARKER 7; SEQ ID NO :7)
AAGAAGAGGTAGCGAGTGGACGTGACTGCTCTATCCCGGGCAAAAGGGATAGAACCAGAG
GTGGGGAGTCTGGGCAGTCGGCGACCCGCGAAGACTTGAGGTGCCGCAGCGGCATCCGGA
GTAGCGCCGGGCTCCCTCCGGGGTGCAGCCGCCGTCGGGGGAAGGGCGCCACAGGCCGGG
AAGACCTCCTCCCTTTGTGTCCAGTAGTGGGGTCCACCGGAGGGCGGCCCGTGGGCCGGG
CCTCACCGCGGCGCTCCGGGACTGTGGGGTCAGGCTGCGTTGGGTGGACGCCCACCTCGC
CAACCTTCGGAGGTCCCTGGGGGTCTTCGTGCGCCCCGGGGCTGCAGAGATCCAGGGGAG
GCGCCTGTGAGGCCCGGACCTGCCCCGGGGCGAAGGGTATGTGGCGAGACAGAGCCCTGC
ACCCCTAATTCCCGGTGGAAAACTCCTGTTGCCGTTTCCCTCCACCGGCCTGGAGTCTCC
CAGTCTTGTCCCGGCAGTGCCGCCCTCCCCACTAAGACCTAGGCGCAAAGGCTTGGCTCA
TGGTTGACAGCTCAGAGAGAGAAAGATCTGAGGGAAGATGGATGCAAAAGCTCGAAATTG
TTTGCTTCAACATAGAGAAGCTCTGGAAAAGGACATCAAGACATCCTACATCATGGATCA
CATGATTAGTGATGGATTTTTAACAATATCAGAAGAGGAAAAAGTAAGAAATGAGCCCAC
TCAACAGCAAAGAGCAGCTATGCTGATTAAAATGATACTTAAAAAAGATAATGATTCCTA
CGTATCATTCTACAATGCTCTACTACATGAAGGATATAAAGATCTTGCTGCCCTTCTCCA
TGATGGCATTCCTGTTGTCTCTTCTTCCAGTGTAAGGACAGTCCTGTGTGAAGGTGGAGT
ACCACAGAGGCCAGTTGTTTTTGTCACAAGGAAGAAGCTGGTGAATGCAATTCAGCAGAA
GCTCTCCAAATTGAAAGGTGAACCAGGATGGGTCACCATACATGGAATGGCAGGCTGTGG
GAAGTCTGTATTAGCTGCAGAAGCTGTTAGAGATCATTCCCTTTTAGAAGGTTGTTTCCC
AGGGGGAGTGCATTGGGTTTCAGTTGGGAAACAAGACAAATCTGGGCTTCTGATGAAACT
GCAGAATCTTTGCACACGGTTGGATCAGGATGAGAGTTTTTCCCAGAGGCTTCCACTTAA
TATTGAAGAGGCTAAAGACCGTCTCCGCATTCTGATGCTTCGCAAACACCCAAGGTCTCT
CTTGATCTTGGATGATGTTTGGGACTCTTGGGTGTTGAAAGCTTTTGACAGTCAGTGTCA
GATTCTTCTTACAACCAGAGACAAGAGTGTTACAGATTCAGTAATGGGTCCTAAATATGT
AGTCCCTGTGGAGAGTTCCTTAGGAAAGGAAAAAGGACTTGAAATTTTATCCCTTTTTGT
TAATATGAAGAAGGCAGATTTGCCAGAACAAGCTCATAGTATTATAAAAGAATGTAAAGG
CTCTCCCCTTGTAGTATCTTTAATTGGTGCACTTTTACGTGATTTTCCCAATCGCTGGGA
GTACTACCTCAAACAGCTTCAGAATAAGCAGTTTAAGAGAATAAGGAAATCTTCGTCTTA
TGATTATGAGGCTCTAGATGAAGCCATGTCTATAAGTGTTGAAATGCTCAGAGAAGACAT
CAAAGATTATTACACAGATCTTTCCATCCTTCAGAAGGACGTTAAGGTGCCTACAAAGGT
GTTATGTATTCTCTGGGACATGGAAACTGAAGAAGTTGAAGACATACTGCAGGAGTTTGT
AAATAAGTCTCTTTTATTCTGTGATCGGAATGGAAAGTCGTTTCGTTATTATTTACATGA
TCTTCAAGTAGATTTTCTTACAGAGAAGAATTGCAGCCAGCTTCAGGATCTACATAAGAA
GATAATCACTCAGTTTCAGAGATATCACCAGCCGCATACTCTTTCACCAGATCAGGAAGA
CTGTATGTATTGGTACAACTTTCTGGCCTATCACATGGCCAGTGCCAAGATGCACAAGGA
ACTTTGTGCTTTAATGTTTTCCCTGGATTGGATTAAAGCAAAAACAGAACTTGTAGGCCC
TGCTCATCTGATTCATGAATTTGTGGAATACAGACATATACTAGATGAAAAGGATTGTGC
AGTCAGTGAGAATTTTCAGGAGTTTTTATCTTTAAATGGACACCTTCTTGGACGACAGCC
ATTTCCTAATATTGTACAACTGGGTCTCTGTGAGCCGGAAACTTCAGAAGTTTATCAGCA
AGCTAAGCTGCAGGCCAAGCAGGAGGTCGATAATGGAATGCTTTACCTGGAATGGATAAA
CAAAAAAAACATCACGAATCTTTCCCGCTTAGTTGTCCGCCCCCACACAGATGCTGTTTA
CCATGCCTGCTTTTCTGAGGATGGTCAGAGAATAGCTTCTTGTGGAGCTGATAAAACCTT
ACAGGTGTTCAAAGCTGAAACAGGAGAGAAACTTCTAGAAATCAAGGCTCATGAGGATGA
AGTGCTTTGTTGTGCATTCTCTACAGATGACAGATTTATAGCAACCTGCTCAGTGGATAA
AAAAGTGAAGATTTGGAATTCTATGACTGGGGAACTAGTACACACCTATGATGAGCACTC
AGAGCAAGTCAATTGCTGCCATTTCACCAACAGTAGTCATCATCTTCTCTTAGCCACTGG
GTCAAGTGACTGCTTCCTCAAACTTTGGGATTTGAATCAAAAAGAATGTCGAAATACCAT
GTTTGGTCATACAAATTCAGTCAATCACTGCAGATTTTCACCAGATGATAAGCTTTTGGC
TAGTTGTTCAGCTGATGGAACCTTAAAGCTTTGGGATGCGACATCAGCAAATGAGAGGAA
AAGCATTAATGTGAAACAGTTCTTCCTAAATTTGGAGGACCCTCAAGAGGATATGGAAGT
GATAGTGAAGTGTTGTTCGTGGTCTGCTGATGGTGCAAGGATAATGGTGGCAGCAAAAAA
TAAAATCTTTCTTTTTGACATTCATACTAGTGGCCTATTGGGAGAAATCCACACGGGCCA
TCACAGCACCATCCAGTACTGTGACTTCTCCCCACAAAACCATTTGGCAGTGGTTGCTTT
GTCCCAGTACTGTGTAGAGTTGTGGAATACAGACTCACGTTCAAAGGTGGCTGATTGCAG
AGGACATTTAAGTTGGGTTCATGGTGTGATGTTTTCTCCTGATGGATCATCATTTTTGAC
ATCTTCTGATGACCAGACAATCAGGCTCTGGGAGACAAAGAAAGTATGTAAGAACTCTGC
TGTAATGTTAAAGCAAGAAGTAGATGTTGTGTTTCAAGAAAATGAAGTGATGGTCCTTGC
AGTTGACCATATAAGACGTCTGCAACTCATTAATGGAAGAACAGGTCAGATTGATTATCT
GACTGAAGCTCAAGTTAGCTGCTGTTGCTTAAGTCCACATCTTCAGTACATTGCATTTGG
AGATGAAAATGGAGCCATTGAGATTTTAGAACTTGTAAACAATAGAATCTTCCAGTCCAG
GTTTCAGCACAAGAAAACTGTATGGCACATCCAGTTCACAGCCGATGAGAAGACTCTTAT
TTCAAGTTCTGATGATGCTGAAATTCAGGTATGGAATTGGCAATTGGACAAATGTATCTT
TCTACGAGGCCATCAGGAAACAGTGAAAGACTTTAGACTCTTGAAAAATTCAAGACTGCT
TTCTTGGTCATTTGATGGAACAGTGAAGGTATGGAATATTATTACTGGAAATAAAGAAAA
AGACTTTGTCTGTCACCAGGGTACAGTACTTTCTTGTGACATTTCTCACGATGCTACCAA
GTTTTCATCTACCTCTGCTGACAAGACTGCAAAGATCTGGAGTTTTGATCTCCTTTTGCC
ACTTCATGAATTGAGGGGCCACAACGGCTGTGTGCGCTGCTCTGCCTTCTCTGTGGACAG
TACCCTGCTGGCAACGGGAGATGACAATGGAGAAATCAGGATATGGAATGTCTCAAACGG
TGAGCTTCTTCATTTGTGTGCTCCGCTTTCAGAAGAAGGAGCTGCTACCCATGGAGGCTG
GGTGACTGACCTTTGCTTTTCTCCAGATGGCAAAATGCTTATCTCTGCTGGAGGATATAT
TAAGTGGTGGAACGTTGTCACTGGGGAATCCTCACAGACCTTCTACACAAATGGAACCAA
TCTTAAGAAAATACACGTGTCCCCTGACTTCAAAACATATGTGACTGTGGATAATCTTGG
TATTTTATATATTTTACAGACTTTAGAATAAAATAGTTAAGCATTAATGTAGTTGAACTT
TTTAAATTTTTGAATTGGAAAAAAATTCTAATGAAACCCTGATATCAACTTTTTATAAAG
CTCTTAATTGTTGTGCAGTATTGCATTCATTACAAAAGTGTTTGTGGTTGGATGAATAAT
ATTAATGTAGCTTTTTCCCAAATGAACATACCTTTAATCTTGTTTTTCATGATCATCATT
AACAGTTTGTCCTTAGGATGCAAATGAAAATGTGAATACATACCTTGTTGTACTGTTGGT
AAAATTCTGTCTTGATGCATTCAAAATGGTTGACATAATTAATGAGAAGAATTTGGAAGA
AATTGGTATTTTAATACTGTCTGTATTTATTACTGTTATGCAGGCTGTGCCTCAGGGTAG
CAGTGGCCTGCTTTTTGAACCACACTTACCCCAAGGGGGTTTTGTTCTCCTAAATACAAT
CTTAGAGGTTTTTTGCACTCTTTAAATTTGCTTTAAAAATATTGTGTCTGTGTGCATAGT
CTGCAGCATTTCCTTTAATTGACTCAATAAGTGAGTCTTGGATTTAGCAGGCCCCCCCAC
CTTTTTTTTTTGTTTTTGGAGACAGAGTCTTGCTTTGTTGCCAGGCTGGAGTGCAGTGGC
GCGATCTCGGCTCACCACAATCGCTGCCTCCTGGGTTCAAGCAATTCTCCTGCCTCAGCC
TCCCGAGTAGCTGGGACTACAGGTGTGCGCACATGCCAGGCTAATTTTTGTATTTTTAGT
AGAGACGGGGTTTCACCATGTTGGCCGGGATGGTCTCGATCTCTTGACCTCATGATCTAC
CCGCCTTGGCCTCCCAAAGTGCTGAGATTACAGGCGTGAGCCACCGTGCCTGGCCAGGCC
CCTTCTCTTTTAATGGAGACAGGGTCTTGCACTATCACCCAGGCTGGAGTGCAGTGGCAT
AATCATACCTCATTGCAGCCTCAGACTCCTGGGTTCAAGCAATCCTCCTGCCTCAGCCTC
CCAAGTAGCTGAGACTGCAGGCACGAGCCACCACACCCAGCTAATTTTTAAGTTTTCTTG
TAGAGACAGGGTCTCACTATGTTGTCTAGGCTGGTCTTGAACTCTTGGCCTCAAGTAATC
CTCCTGCCTCAGCCTCCCAAAGTGTTGGGATTGCAGATATGAGCCACTGGCCTGGCCTTC
AGCAGTTCTTTTTGTGAAGTAAAACTTGTATGTTGGAAAGAGTAGATTTTATTGGTCTAC
CCTTTTCTCACTGTAGCTGCTGGCAGCCCTGTGCCATATCTGGACTCTAGTTGTCAGTAT
CTGAGTTGGACACTATTCCTGCTCCCTCTTGTTTCTTACATATCAGACTTCTTACTTGAA
TGAAACCTGATCTTTCCTAATCCTCACTTTTTTCTTTTTTAAAAAGCAGTTTCTCCACTG
CTAAATGTTAGTCATTGAGGTGGGGCCAATTTTAATCATAAGCCTTAATAAGATTTTTCT
AAGAAATGTGAAATAGAACAATTTTCATCTAATTCCATTTACTTTTAGATGAATGGCATT
GTGAATGCCATTCTTTTAATGAATTTCAAGAGAATTCTCTGGTTTTCTGTGTAATTCCAG
ATGAGTCACTGTAACTCTAGAAGATTAACCTTCCAGCCAACCTATTTTCCTTTCCCTTGT
CTCTCTCATCCTCTTTTCCTTCCTTCTTTCCTTTCTCTTCTTTTATCTCCAAGGTTAATC
AGGAAAAATAGCTTTTGACAGGGGAAAAAACTCAATAACTAGCTATTTTTGACCTCCTGA
TCAGGAACTTTAGTTGAAGCGTAAATCTAAAGAAACATTTTCTCTGAAATATATTATTAA
GGGCAATGGAGATAAATTAATAGTAGATGTGGTTCCCAGAAAATATAATCAAAATTCAAA
GATTTTTTTTGTTTCTGTAACTGGAACTAAATCAAATGATTACTAGTGTTAATAGTAGAT
AACTTGTTTTTATTGTTGGTGCATATTAGTATAACTGTGGGGTAGGTCGGGGAGAGGGTA
AGGGAATAGATCACTCAGATGTATTTTAGATAAGCTATTTAGCCTTTGATGGAATCATAA
ATACAGTGAATACAATCCTTTGCATTGTTAAGGAGGTTTTTTGTTTTTAAATGGTGGGTC
AAGGAGCTAGTTTACAGGCTTACTGTGATTTAAGCAAATGTGAAAAGTGAAACCTTAATT
TTATCAAAAGAAATTTCTGTAAATGGTATGTCTCCTTAGAATACCCAAATCATAATTTTA
TTTGTACACACTGTTAGGGGCTCATCTCATGTAGGCAGAGTATAAAGTATTACCTTTTGG
AATTAAAAGCCACTGACTGTTATAAAGTATAACAACACACATCAGGTTTTAAAAAGCCTT
GAATGGCCCTTGTCTTAAAAAGAAATTAGGAGCCAGGTGCGGTGGCACGTGCCTGTAGTC
CCAGCTCCTTGGGAGGCTGAGACAGGAGGATTCCTTGAGCCCTGGAGTTTGAGTCCAGCC
TGGGTGACATAGCAAGACCCTGTCTTAAAAGAAAAATGGGAAGAAAGACAAGGTAACATG
AAGAAAGAAGAGATACCTAGTATGATGGAGCTGCAAATTTCATGGCAGTTCATGCAGTCG
GTCAAGAGGAGGATTTTGTTTTGTAGTTTGCAGATGAGCATTTCTAAAGCATTTTCCCTT
GCTGTATTTTTTTGTATTATAAATTACATTGGACTTCATATATATAATTTTTTTTTACAT
TATATGTCTCTTGTATGTTTTGAAACTCTTGTATTTATGATATAGCTTATATGATTTTTT
TGCCTTGGTATACATTTTAAAATATGAATTTAAAAAATTTTTGTAAAAATAAAATTCACA
AAATTGTTTTGAAAAACAAAAAAAAAAAAAA
ERMAP (erythroblast membrane-associated protein)
(PSYCHMARKER 8; SEO ID NO :8)
ATGGAGATGG CGAGTTCTGC TGGCTCCTGG CTCTCTGGCT GCCTCATCCC TCTCGTCTTC
CTCCGGCTGT CTGTGCATGT GTCAGGCCAC GCAGGGGATG CCGGCAAGTT CCACGTGGCC
CTACTAGGGG GCACAGCCGA GCTGCTCTGC CCTCTCTCCC TCTGGCCCGG GACGGTACCC
AAGGAGGTGA GGTGGCTGCG GTCCCCATTC CCGCAGCGTT CCCAGGCTGT TCACATATTC
CGGGATGGGA AGGACCAGGA TGAAGATCTG ATGCCGGAAT ATAAGGGGAG GACGGTGCTA
GTGAGAGATG CCCAAGAGGG AAGTGTCACT CTGCAGATCC TTGACGTGCG CCTTGAGGAC
CAAGGGTCTT ACCGATGTCT GATCCAAGTT GGAAATCTGA GTAAAGAGGA CACCGTGATC
CTGCAGGTTG CAGCCCCATC TGTGGGGAGT CTCTCCCCCT CAGCAGTGGC TCTGGCTGTG
ATCCTGCCTG TCCTGGTACT TCTCATCATG GTGTGCCTTT GCCTTATCTG GAAGCAAAGA
AGAGCAAAAG AAAAGCTTCT CTATGAACAT GTGACGGAGG TGGACAATCT TCTTTCAGAC
CATGCTAAAG AAAAAGGAAA ACTCCATAAA GCTGTCAAGA AACTCCGGAG TGAACTGAAG
TTGAAAAGAG CTGCAGCAAA CTCAGGCTGG AGAAGAGCCC GGTTGCATTT TGTGGCAGTG
ACCCTGGACC CAGACACAGC ACATCCCAAA CTCATCCTTT CTGAGGACCA AAGATGTGTA
AGGCTTGGAG ACAGACGGCA GCCTGTACCT GACAACCCCC AGAGATTTGA TTTCGTTGTC
AGCATCCTAG GCTCTGAGTA CTTCACGACT GGCTGCCACT ACTGGGAGGT GTATGTGGGA
GACAAGACCA AATGGATTCT TGGAGTATGT AGTGAGTCAG TGAGCAGGAA GGGGAAGGTT
ACTGCCTCAC CTGCCAATGG ACACTGGCTT CTGCGACAGA GTCGTGGGAA TGAGTATGAA
GCTCTCACAT CCCCGCAGAC CTCCTTCCGC CTTAAAGAGC CTCCACGGTG TGTGGGGATT
TTCCTGGACT ATGAAGCAGG AGTCATCTCT TTCTACAATG TGACCAACAA GTCCCACATC
TTTACTTTCA CCCACAATTT CTCTGGCCCC CTTCGCCCTT TCTTTGAACC TTGCCTTCAT
GATGGAGGAA AAAACACAGC ACCTCTAGTC ATTTGTTCAG AACTACACAA ATCAGAGGAA
TCAATTGTCC CCAGGCCAGA AGGGAAAGGC CATGCTAATG GAGATGTGTC CCTCAAGGTG
AACTCTTCTT TACTACCCCC GAAGGCCCCA GAGCTGAAGG ATATAATCCT GTCCTTGCCC
CCTGACCTTG GCCCAGCCCT TCAGGAGCTC AAGGCTCCTT CTTTTTAG
CGFLC 31120 (PSYCHMARKER 9: SEQ ID NO :9)
ACCGGTAATG ACCTCCGCGA TACCATACAC CATTGAGGTT GGAGTGGGCG CAGGCATGGT
ACCACCATCC CCCACGATGG TAGAGGGCAC AGTTGCCTGA GGGGGGAGAG AGAGGAGAGA
GAAAGAGACA GCATTCGATT TCCCCCCAAA ATAAAAGGAT CTACCAGCAC TTTCTTTGGG
CAAAGCTT
LYPLA1 lysophospholipase 1 (PSYCHMARKER 10; SEQ ID NO :10)
AGCCGCTCGCACGCCCTTGGGCCGCGGCCGGGCGCCCGCTCTTCCTTCCGCTTGCGCTGT
GAGCTGAGGCGGTGTATGTGCGGCAATAACATGTCAACCCCGCTGCCCGCCATCGTGCCC
GCCGCCCGGAAGGCCACCGCTGCGGTGATTTTCCTGCATGGATTGGGAGATACTGGGCAC
GGATGGGCAGAAGCCTTTGCAGGTATCAGAAGTTCACATATCAAATATATCTGCCCGCAT
GCGCCTGTTAGGCCTGTTACATTAAATATGAACGTGGCTATGCCTTCATGGTTTGATATT
ATTGGGCTTTCACCAGATTCACAGGAGGATGAATCTGGGATTAAACAGGCAGCAGAAAAT
ATAAAAGCTTTGATTGATCAAGAAGTGAAGAATGGCATTCCTTCTAACAGAATTATTTTG
GGAGGGTTTTCTCAGGGAGGAGCTTTATCTTTATATACTGCCCTTACCACACAGCAGAAA
CTGGCAGGTGTCACTGCACTCAGTTGCTGGCTTCCACTTCGGGCTTCCTTTCCACAGGGT
CCTATCGGTGGTGCTAATAGAGATATTTCTATTCTCCAGTGCCACGGGGATTGTGACCCT
TTGGTTCCCCTGATGTTTGGTTCTCTTACGGTGGAAAAACTAAAAACATTGGTGAATCCA
GCCAATGTGACCTTTAAAACCTATGAAGGTATGATGCACAGTTCGTGTCAACAGGAAATG
ATGGATGTCAAGCAATTCATTGATAAACTCCTACCTCCAATTGATTGACGTCACTAAGAG
GCCTTGTGTAGAAGTACACCAGCATCATTGTAGTAGAGTGTAAACCTTTTCCCATGCCCA
GTCTTCAAATTTCTAATGTTTTGCAGTGTTAAAATGTTTTGCAAATACATGCCAATAACA
CAGATCAAATAATATCTCCTCATGAGAAATTTATGATCTTTTAAGTTTCTATACATGTAT
TCTTATAAGACGACCCAGGATCTACTATATTAGAATAGATGAAGCAGGTAGCTTCTTTTT
TCTCAAATGTAATTCAGCAAAATAATACAGTACTGCCACCAGATTTTTTATTACATCATT
TGAAAATTAGCAGTATGCTTAATGAAAATTTGTTCAGGTATAAATGAGCAGTTAAGATAT
AAACAATTTATGCATGCTGTGACTTAGTCTATGGATTTATTCCAAAATTGCTTAGTCACC
ATGCAGTGTCTGTATTTTTATATATGTGTTCATATATACATAATGATTATAATACATAAT
AAGAATGAGGTGGTATTACATTATCCCTAATAATAGGGATAATGCTGNTTATTGTCCAGG
AAAAAGTAAAATCGGTCCCCTTCAATTAATGGCCCTTTTAATNTNGGGACCAGGCTTTTA
ATTTTCCCCGGATATTAATTTCCAATTTAATACCCCTTTCCNCNCCAGAAAAAAAAAAAA
AGTTTGTTTTTTCCTTAATTGTCTTCATAGCAGGCCAAGTATTGCC
LDHA Lactate dehydrogenase A (PSYCHMARKER 11; SEQ ID NO :11)
ATGGCAACTC TAAAGGATCA GCTGATTTAT AATCTTCTAA AGGAAGAACA GACCCCCCAG
AATAAGATTA CAGTTGTTGG GGTTGGTGCT GTTGGCATGG CCTGTGCCAT CAGTATCTTA
ATGAAGGACT TGGCAGATGA ACTTGCTCTT GTTGATGTCA TCGAAGACAA ATTGAAGGGA
GAGATGATGG ATCTCCAACA TGGCAGCCTT TTCCTTAGAA CACCAAAGAT TGTCTCTGGC
AAAGACTATA ATGTAACTGC AAACTCCAAG CTGGTCATTA TCACGGCTGG GGCACGTCAG
CAAGAGGGAG AAAGCCGTCT TAATTTGGTC CAGCGTAACG TGAACATATT TAAATTCATC
ATTCCTAATG TTGTAAAATA CAGCCCGAAC TGCAAGTTGC TTATTGTTTC AAATCCAGTG
GATATCTTGA CCTACGTGGC TTGGAAGATA AGTGGTTTTC CCAAAAACCG TGTTATTGGA
AGTGGTTGCA ATCTGGATTC AGCCCGATTC CGTTACCTGA TGGGGGAAAG GCTGGGAGTT
CACCCATTAA GCTGTCATGG GTGGGTCCTT GGGGAACATG GAGATTCCAG TGTGCCTGTA
TGGAGTGGAA TGAATGTTGC TGGTGTCTCT CTGAAGACTC TGCACCCAGA TTTAGGGACT
GATAAAGATA AGGAACAGTG GAAAGAGGTT CACAAGCAGG TGGTTGAGAG TGCTTATGAG
GTGATCAAAC TCAAAGGCTA CACATCCTGG GCTATTGGAC TCTCTGTAGC AGATTTGGCA
GAGAGTATAA TGAAGAATCT TAGGCGGGTG CACCCAGTTT CCACCATGAT TAAGGGTCTT
TACGGAATAA AGGATGATGT CTTCCTTAGT GTTCCTTGCA TTTTGGGACA GAATGGAATC
TCAGACCTTG TGAAGGTGAC TCTGACTTCT GAGGAAGAGG CCCGTTTGAA GAAGAGTGCA
GATACACTTT GGGGGATCCA AAAGGAGCTG CAATTTTAA
SCG3 (secretogranin III) (PSYCHMARKER 12; SEO ID NO :12)
ATGGGGTTCC TCGGGACCGG CACTTGGATT CTGGTGTTAG TGCTCCCGAT TCAAGCTTTC
CCCAAACCTG GAGGAAGCCA AGACAAATCT CTACATAATA GAGAATTAAG TGCAGAAAGA
CCTTTGAATG AACAGATTGC TGAAGCAGAA GAAGACAAGA TTAAAAAAAC ATATCCTCCA
GAAAACAAGC CAGGTCAGAG CAACTATTCT TTTGTTGATA ACTTGAACCT GCTAAAGGCA
ATAACAGAAA AGGAAAAAAT TGAGAAAGAA AGACAATCTA TAAGAAGCTC CCCACTTGAT
AATAAGTTGA ATGTGGAAGA TGTTGATTCA ACCAAGAATC GAAAACTGAT CGATGATTAT
GACTCTACTA AGAGTGGATT GGATCATAAA TTTCAAGATG ATCCAGATGG TCTTCATCAA
CTAGACGGGA CTCCTTTAAC CGCTGAAGAC ATTGTCCATA AAATCGCTGC CAGGATTTAT
GAAGAAAATG ACAGAGCCGT GTTTGACAAG ATTGTTTCTA AACTACTTAA TCTCGGCCTT
ATCACAGAAA GCCAAGCACA TACACTGGAA GATGAAGTAG CAGAGGTTTT ACAAAAATTA
ATCTCAAAGG AAGCCAACAA TTATGAGGAG GATCCCAATA AGCCCACAAG CTGGACTGAG
AATCAGGCTG GAAAAATACC AGAGAAAGTG ACTCCAATGG CAGCAATTCA AGATGGTCTT
GCTAAGGGAG AAAACGATGA AACAGTATCT AACACATTAA CCTTGACAAA TGGCTTGGAA
AGGAGAACTA AAACCTACAG TGAAGACAAC TTTGAGGAAC TCCAATATTT CCCAAATTTC
TATGCGCTAC TGAAAAGTAT TGATTCAGAA AAAGAAGCAA AAGAGAAAGA AACACTGATT
ACTATCATGA AAACACTGAT TGACTTTGTG AAGATGATGG TGAAATATGG AACAATATCT
CCAGAAGAAG GTGTTTCCTA CCTTGAAAAC TTGGATGAAA TGATTGCTCT TCAGACCAAA
AACAAGCTAG AAAAAAATGC TACTGACAAT ATAAGCAAGC TTTTCCCAGC ACCATCAGAG
AAGAGTCATG AAGAAACAGA CAGTACCAAG GAAGAAGCAG CTAAGATGGA AAAGGAATAT
GGAAGCTTGA AGGATTCCAC AAAAGATGAT AACTCCAACC CAGGAGGAAA GACAGATGAA
CCCAAAGGAA AAACAGAAGC CTATTTGGAA GCCATCAGAA AAAATATTGA ATGGTTGAAG
AAACATGACA AAAAGGGAAA TAAAGAAGAT TATGACCTTT CAAAGATGAG AGACTTCATC
AATAAACAAG CTGATGCTTA TGTGGAGAAA GGCATCCTTG ACAAGGAAGA AGCCGAGGCC
ATCAAGCGCA TTTATAGCAG CCTGTAA
CG187232-01 (hypothetical protein)
(PSYCHMARKER 13; SEQ ID NO :13)
CAATAGAAATGTTTGGCTTTACCCATCAGCCAAATAAAAAAATCTCCTTGTAAGGTTAAA
AGGATCAAAATGTAGGAGACCGTGAATATTCACCAAAACCCTTCATCTTTTCAATTTCTT
CATCTGTTTCTGGGCTGCTCATTTTGGATGCTTTATCTTGGTTACTACTACCAGTATTTG
CTTTAGATCGTTTGTCTTGTGTATCTAACAACCGAGATCTCTTGAATTGCCATCTGTCAT
CTAAGTCTAAGGCTTTATGCTTAATGATTTGTGGTGTACTAACTGGACTAGCTTCAGGAA
TGTCAGTGTGTTCTACCATTAAGGTCAGATTGTCTACTACATCGAGATGGTGGTTGTAGT
TACAGCTACTTTTAGAAACATGTCTATTTTTTAAAGACATAACACATGAATGAAGAAATT
CAGAATTTGGAAAAAAGGTCCGTAATGCCTGACAAAGAAAAATGTTCTCCTGAGGGTCTT
TTTGGATGTTCTTCTCTCTTGCTGCCTGAATCTGTGCTCCTCTCCTTTTCTCAATTTCTC
GTTTTAATCTGTTTACATCCTTTACTAGTTTATCTACTGCTTGTTCACTGTCTTCCACTT
TTTTGCATATACTCCTTAATTCCTCTTGTAATGAAGCATACATTTCATTTATCTTATGTA
CCTCCTTTAAGGATCCATCTTCTTCAAAAAATTTAGAGCTGTGTGTTTGTACTGCTCGGC
TAAAACCAGTGGACATACAGGAACCTGATACAGTTTTATAACCCAGTTGTTCAGACATGC
CCAGATTGGCAACCACTAAAGGTACCCTGTGAAAAAGTCCTTTTTGAGGTTTATATAAGG
AATGTTCCAGTCGATGAGTAGAGCAGCTTTCTGTTATTATACTTGGTGTTAATAGCAGAA
AAACAAGGTCTTGGTTTGAAAAATGCTCCTGCAAGTTTTTGTGAAGCAGCCTCTCTCTAA
ACGTCATGATCTGATCTGAATGACGACGGAATTTGTACCAACCTACCACATTCTTTTTGA
CATTTGATAATATTTTCTTCAGTGCTTGCTCATTTACTTCG

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of identifying a psychoactive compound, comprising contacting a cell with a test compound and determining a level of expression of a psychoactive-associated gene in the cell, wherein an increase or decrease of said level compared to a normal control level of said gene indicates that the test compound is a psychoactive compound

2. The method of claim 1, wherein said psychoactive-associated gene is selected from the group consisting of PTX3, ILK, ENTPD6, GPCR CG50207, SFRS7, CBRC7TM_{—}424 GPCR, APAF-1, ERMAP, CGFLC_{—}31120, LYPLA1, LDHA, SCG3 and CG187132.

3. The method of claim 1, wherein said method further comprises determining said level of expression of a plurality of psychoactive-associated genes.

4. The method of claim 1, wherein said psychoactive compound is an antidepressant compound, an antipsychotic compound or an opioid compound.

5. A psychoactive compound reference expression profile, comprising a pattern of gene expression of two or more genes selected from the group consisting of PTX3, ILK, ENTPD6, GPCR CG50207, SFRS7, CBRC7TM_{—}424 GPCR, APAF_{—}1, ERMAP,
CGFLC_{—}31120, LYPLA1, LDHA, SCG3 and CG187132.

6. A method of identifying an agent that inhibits the expression or activity of a psychoactive-associated, comprising contacting a test cell expressing said psychoactive-associated gene with a test agent and determining the expression level of said psychoactive-associated gene, wherein a decrease of said level compared to a normal control level of said gene indicates that said test agent is an inhibitor of said psychoactive-associated gene.

7. A method of identifying an agent that enhances the expression or activity of a psychoactive-associated gene, comprising contacting a test cell expressing said psychoactive-associated gene with a test agent and determining the expression level or activity of the psychoactive-associated gene wherein an increase of said level or activity compared to a normal control level or activity of said gene indicates that said test agent is an enhancer of the psychoactive-associated gene.

8. An array comprising a plurality of oligonucleotides which binds
a. the nucleic acid sequences of SEQ ID NO : 1-13 or
b. the nucleic acid sequences of at least four nucleic acid sequences selected from the group consisting of SEQ ID NO : 1-13.

9. A kit comprising a detection reagent that identifies:
a. the nucleic acid sequences of SEQ ID NO : 1-13 or
b. the nucleic acid sequences of at least four nucleic acid sequences selected from the group consisting of SEQ ID NO : 1-13.
